(19) United States
(12) Patent Application Publication
(10) Pub. No.: US 2026/0183261 A1
(43) Pub. Date: Jul. 2, 2026

(54) Compound or pharmaceutically acceptable salts thereof for use in rejuvenation composition of aged microglia and pharmaceutical composition for prevention or treatment of brain diseases containing the same composition for preventing or treating brain diseases containing the same

(71) Applicants: Julia Laboratory, Suwon-si (KR); Brainimmunex Inc., Suwon-si (KR)

(72) Inventors: Wan Jin JAHNG, Suwon-si (KR); Kyung Hae LEE, Suwon-si (KR); Min-Soo KWON, Seoul (KR)

(21) Appl. No.: 19/411,355

(22) Filed: Dec. 7, 2025

(30) Foreign Application Priority Data
Dec. 27, 2024 (KR) 10-2024-0198422
Mar. 4, 2025 (KR) 10-2025-0027373

(51) Int. Cl. A61K 31/401; A61K 31/427; A61P 25/28

(52) U.S. Cl. CPC A61K 31/401; A61K 31/427; A61P 25/28

(57) ABSTRACT

Provided is to a compound or a pharmaceutically acceptable salt thereof for use in a composition for rejuvenating aged microglia, and a pharmaceutical composition for preventing or treating brain diseases containing the same. Also disclosed is the use of a compound represented by Formula 1 or a pharmaceutically acceptable salt thereof to restore the phagocytic function of microglia that has been impaired by dexamethasone, thereby providing a rejuvenation composition for aged microglia that can be usefully applied as a pharmaceutical composition for preventing or treating brain diseases including neurodegenerative diseases, neuroinflammatory diseases, depression, and neuropsychiatric disorders.

### BACKGROUND OF THE INVENTION

### Field of the Invention

The present invention relates to a compound or a pharmaceutically acceptable salt thereof for use in a composition for rejuvenating aged microglia, and a pharmaceutical composition for preventing or treating brain diseases containing the same. More specifically, the compound represented by Formula 1 or a pharmaceutically acceptable salt thereof restores the phagocytic function of microglia impaired by dexamethasone, thereby being useful in a rejuvenation composition for aged microglia and furthermore being pharmaceutically useful for preventing or treating brain diseases including neurodegenerative diseases, neuroinflammatory diseases, and neuropsychiatric disorders including depression.

### Description of the Related Art

Cells, the basic units constituting living organisms, replicate themselves during the cell cycle and divide into two identical cells. Every moment we live, cells die and divide repeatedly, maintaining form and enabling growth. However, cell division has limitations, and cells that exceed this limit become senescent cells, displaying unique properties. The accumulation of senescent cells triggers many inflammatory responses and age-related diseases.

Not only neurons, which are postmitotic cells that no longer divide and have completed differentiation, but also cells that develop and die during neurodevelopment can exhibit characteristics of senescent cells due to external environments and stimuli.

As we enter a super-aged society, along with life extension, the incidence of degenerative brain diseases such as Alzheimer's disease, Parkinson's disease, and ALS (Lou Gehrig's disease) is rapidly increasing. One of the major causes of these diseases is the accumulation of toxic proteins in the brain (e.g., amyloid-beta, tau protein, alpha-synuclein, TDP-43), and their effective removal is considered one of the key factors in maintaining brain health.

Microglia are the representative innate immune cells present in the brain. They are responsible for synaptic pruning during neurogenesis in the brain. Synaptic pruning is a part of neural development where excessively generated synapses in early development are selectively eliminated by neural activity, leaving only the necessary parts. If this process occurs excessively with age, it can cause cognitive dysfunction due to synaptic dendritic reduction in neurons.

Conversely, as aging progresses, a decrease in phagocytosis, which removes toxic proteins such as amyloid-beta (Aβ), a major cause of Alzheimer's disease, alpha-synuclein associated with Parkinson's disease, and TDP-43 associated with Lou Gehrig's disease, accelerates the accumulation of toxic proteins and increases brain inflammatory responses, leading to neuronal damage.

According to Non-Patent Document 1, it has been reported that as microglia become senescent, their phagocytic function decreases and toxic proteins are not removed, thereby inducing brain aging and neurodegenerative diseases such as Alzheimer's, Parkinson's, and Lou Gehrig's diseases, as well as neuroinflammation and cognitive impairment.

Unlike neurons in the brain, glial cells such as microglia do undergo cell division, but this occurs very rapidly in pathological states and progresses very slowly under normal conditions. Along with reports that microglial senescence reduces phagocytic function, aged microglia isolated from aged brains have been observed to fail in removing amyloid-beta.

Therefore, the accumulation of aged microglia accelerates brain aging and increases vulnerability to neurodegenerative diseases. Research is underway on approaches to remove aged microglia or restore their function.

Non-patent Document 2 presents a method using nanoparticles to deliver microglia-targeted genomes in an Alzheimer's dementia animal model, reporting that inhibiting the senescence-inducing factor p16ink4a gene reverses senescent microglia into young-like microglia, improving phagocytic function and cognitive function.

Patent Document 1 proposes a method to reduce inflammation associated with neurological or cognitive decline in subjects by inhibiting EP2 (prostaglandin E2 receptor 2)-mediated signaling by contacting EP2 with EP2 antagonists. Specifically, it reports that in aged mice, bone marrow cell bioenergetics are suppressed in response to increased signaling of the lipid mediator prostaglandin E2 (PGE2), a major regulator of inflammation, and that PGE2 signaling through EP2 receptors in aged macrophages and microglia sequesters glucose as glycogen, reducing glucose flux and mitochondrial respiration. It also reports that inhibition of myeloid EP2 signaling restores youthful energy metabolism in peripheral macrophages and microglia, recovers systemic and brain inflammatory states, and prevents loss of hippocampal synaptic plasticity and spatial memory.

Therefore, the present inventors focused on the fact that most degenerative brain diseases are geriatric diseases, that is, the fundamental causes are the spread of aging to adjacent cells due to microglia aging and the accumulation of toxic proteins due to reduced phagocytic function of microglia. The present inventors completed the present invention by confirming the results of lowering the cytotoxicity of aged microglia, lowering the expression of the senescence marker SA-j-gal, and simultaneously recovering the phagocytic function of brain immune cells to the pre-aging state by using a novel compound having a low molecular weight capable of penetrating the blood-brain barrier (BBB).

### RELATED ART DOCUMENTS

(Patent Document 1) US Patent Publication No. 2022-0048987 (Published Feb. 17, 2022)

### Non-Patent Documents

(Non-Patent Document 1) Transl Neurodegener 2024 Feb. 20; 13(1):10. Emerging role of senescent microglia in brain aging-related neurodegenerative diseases
(Non-Patent Document 2) Mol. Neurodegener., 2024 Mar. 16, 19(1) Rejuvenating aged microglia by p16ink4a-siRNA-loaded nanoparticles increases amyloid-β clearance in animal models of Alzheimer's disease

### SUMMARY OF THE INVENTION

An object of the present invention is to provide a novel compound or a pharmaceutically acceptable salt thereof.

Another object of the present invention is to provide a composition for the rejuvenation of aged microglia containing a novel compound.

Another object of the present invention is to provide a pharmaceutical composition for preventing or treating brain diseases containing a novel compound.

To achieve the above object, the present invention provides a compound represented by Formula 1 below or a pharmaceutically acceptable salt thereof for use in a composition for rejuvenating aged microglia;

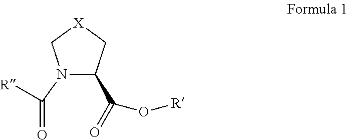

wherein, in Formula 1,
R′ is hydrogen, phenyl, benzyl, or C₁-C₄ linear or branched alkyl;
R″ is a C₁-C₆ alkyl group unsubstituted or substituted with hydroxy (—OH), halogen, C₁-C₄ alkoxy, C₁-C₄ alkylamino or di(C₁-C₄) alkylamino, alkoxycarbonyl, phenyl, phenoxy, or a 5- to 6-membered heteroaryl containing N, O, or S; and
X is any one selected from the group consisting of CH₂—, —S—, —O—, —NH—, or —N(C₁-C₄ alkyl) and —C═O (carbonyl).

The compound of the present invention has a low molecular weight capable of penetrating the Blood-Brain Barrier (BBB) and is preferably a compound having a weight average molecular weight (MW) of 800 g/mol or less.

The compound represented by Formula 1 or pharmaceutically acceptable salt thereof realizes the recovery of phagocytic function of aged microglia induced by dexamethasone.

Accordingly, the present invention provides a rejuvenation composition for aged microglia containing the compound represented by Formula 1 or a pharmaceutically acceptable salt thereof. Preferably, the compound represented by Formula 1 or a pharmaceutically acceptable salt thereof is contained at a concentration of 0.1 to less than 5 mM.

The present invention also provides a pharmaceutical composition for preventing or treating brain diseases selected from the group consisting of neurodegenerative diseases, neuroinflammatory diseases, and neuropsychiatric disorders including depression, containing the compound represented by Formula 1 or pharmaceutically acceptable salt thereof.

More preferably, the pharmaceutical composition can be usefully applied to any brain disease selected from the group consisting of Alzheimer's Disease, Parkinson's Disease, Huntington's Disease, ALS, frontotemporal dementia, Multiple Sclerosis, and Stroke.

### Advantageous Effects

The present invention can provide a novel compound having a low molecular weight capable of penetrating the blood-brain barrier (BBB), which is advantageous for studying the aging mechanism of microglia.

By using the novel compound of the present invention to confirm the performance of recovering the function of microglia degraded by dexamethasone, particularly by lowering the cytotoxicity of aged microglia and lowering the expression of the senescence marker SA-β-gal while simultaneously recovering the phagocytic function of brain immune cells to the pre-aging state, a composition for rejuvenating aged microglia can be provided.

Furthermore, based on the recovery of the phagocytic function of aged microglia by the novel compound of the present invention, it can be usefully applied as a pharmaceutical composition for preventing or treating brain diseases including neurodegenerative diseases, neuroinflammatory diseases, depression, and neuropsychiatric diseases.

### BRIEF DESCRIPTION OF THE DRAWINGS

FIG. 1 shows the cytotoxicity experimental results of primary cultured microglia using the compounds of Example 1 and Example 2 of the present invention.

FIG. 2 shows the cytotoxicity experimental results of primary cultured microglia using the compounds of Example 3 and Example 4 of the present invention.

FIG. 3 shows images of control and experimental groups according to SA-β-gal staining for the compounds of the present invention.

FIG. 4 shows the quantitative results according to SA-β-gal staining by concentration of the compounds of Example 1 and Example 2 of the present invention.

FIG. 5 shows the quantitative results according to SA-β-gal staining by concentration of the compounds of Example 3 and Example 4 of the present invention.

FIG. 6 shows images by experimental group for the brain immune cell phagocytic function recovery experiment for the compounds of the present invention.

FIG. 7 shows the results on brain immune cell phagocytic function recovery by concentration of Examples 1 and 2 compounds of the present invention

FIG. 8 shows the results of brain immune cell phagocytic function recovery by concentration of the compound of Example 3 of the present invention.

FIG. 9 is a live image of phagocytic function for zymosan by concentration of the compound of Example 2 of the present invention.

FIG. 10 shows the results of quantifying the phagocytic intensity over time for zymosan in FIG. 9.

FIG. 11 shows oxygen consumption rate (OCR) results through Seahorse analysis by Example 2 compound of the present invention.

FIG. 12 shows the results on mitochondrial function recovery through the Seahorse analysis of FIG. 11.

FIG. 13 shows the results of changes in cell metabolic function (ECAR) through cell metabolism analysis by the compound of Example 2 of the present invention.

FIG. 14 shows quantitative results related to basic glycolysis from the analysis of FIG. 13.

### DETAILED DESCRIPTION OF THE EMBODIMENTS

Hereinafter, the present invention will be described in detail.

The present invention provides a compound represented by the following Formula 1 or a pharmaceutically acceptable salt thereof for use in a for use in a composition for rejuvenating aged microglia.

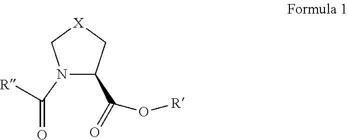

In Formula 1, R′ is hydrogen, phenyl, benzyl, or C₁-C₄ linear or branched alkyl;
R″ is a C₁-C₆ alkyl group unsubstituted or substituted with hydroxy (—OH), halogen, C₁-C₄ alkoxy, C₁-C₄ alkylamino or di(C₁-C₄) alkylamino, alkoxycarbonyl, phenyl, phenoxy, or a 5- to 6-membered heteroaryl containing N, O, or S; and
X is any one selected from the group consisting of CH₂—, —S—, —O—, —NH—, or —N(C₁-C₄ alkyl) and —C═O (carbonyl).

The compound represented by Formula 1 of the present invention is designed as a low molecular weight compound capable of penetrating the blood-brain barrier (BBB) to utilize a delivery system that can selectively target microglia, with a weight average molecular weight (MW) of 800 g/mol or less, preferably 500 g/mol or less, more preferably 350 g/mol or less.

In one example of the present invention, the compound represented by Formula 1 can be used in the form of a pharmaceutically acceptable salt, wherein acid addition salts formed by pharmaceutically acceptable free acids are useful. Free acids include inorganic acids such as hydrochloric acid, sulfuric acid, nitric acid, phosphoric acid, hydrobromic acid, hydroiodic acid; organic carboxylic acids such as tartaric acid, formic acid, citric acid, acetic acid, trichloroacetic acid, trifluoroacetic acid, gluconic acid, benzoic acid, lactic acid, mandelic acid, fumaric acid, maleic acid, salicylic acid; or sulfonic acids such as methanesulfonic acid, ethanesulfonic acid, benzenesulfonic acid, p-toluenesulfonic acid, but are not limited thereto.

Additionally, the compound represented by Formula 1 of the present invention includes not only pharmaceutically acceptable salts but also stereoisomers, solvates, and hydrates that can be prepared by conventional methods.

The addition salt according to the present invention can be prepared by a conventional method, for example, by dissolving the compound of Formula 1 in a water-miscible organic solvent, for example, acetone, methanol, ethanol, or acetonitrile, adding an excess of organic acid or an aqueous acid solution of inorganic acid, and then precipitating or crystallizing. Subsequently, the solvent or excess acid can be evaporated from the mixture and dried to obtain the addition salt, or the precipitated salt can be prepared by suction filtration.

Hereinafter, for efficacy testing of the compound represented by Formula 1 or pharmaceutically acceptable salt thereof, the neuroepithelial layer was isolated from 13.5-day embryos of B6 mice and cultured for 21 days, then microglia were selectively isolated to prepare pure microglia as the control group (YOUNG), and an experimental group (Senescent) of aged microglia induced by dexamethasone was prepared to explain experimental results on the recovery of phagocytic function of aged microglia by the compound-treated groups.

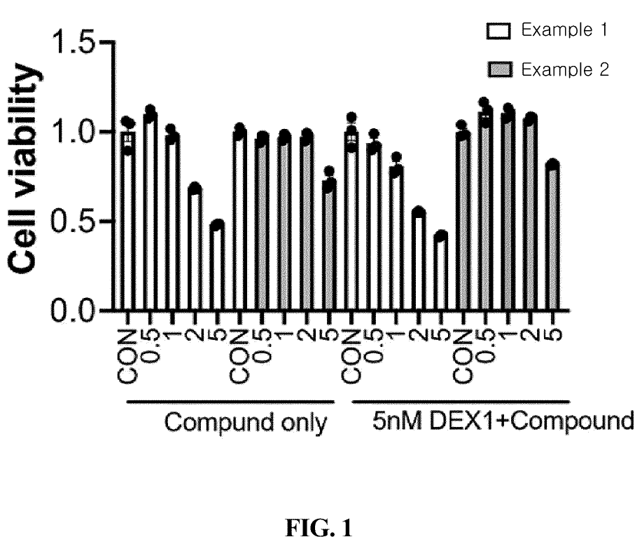

FIG. 1 shows the cytotoxicity experimental results of primary cultured microglia using the compounds of Example 1 and Example 2 of the present invention. The Example 1 and Example 2 compounds partially recovered the decreased viability of aged microglia induced by 5 nM dexamethasone, with particularly confirmed increasing viability trends in the concentration range of 0.5 to 2 mM.

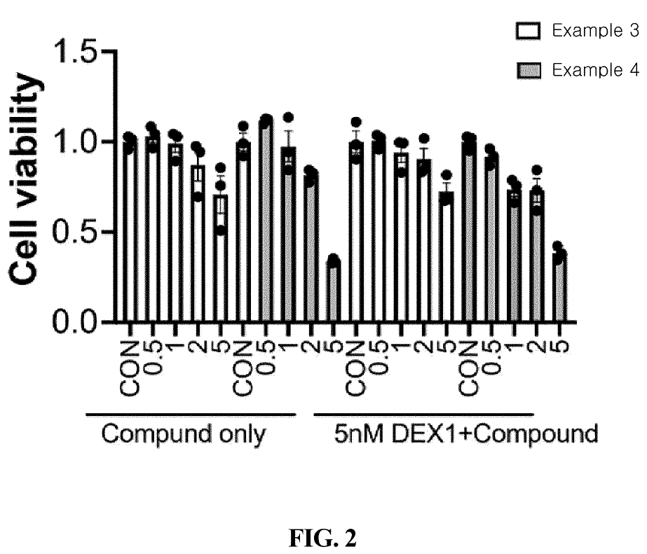

FIG. 2 shows the cytotoxicity experimental results of primary cultured microglia using the compounds of Example 3 and Example 4 of the present invention. Similarly, Examples 3 and 4 compounds also support having cell viability increasing effects while exhibiting low cytotoxicity. From the above, it can be confirmed that the compounds of Examples 1 through 4 of the present invention have functions to alleviate cellular stress on aged microglia induced by dexamethasone.

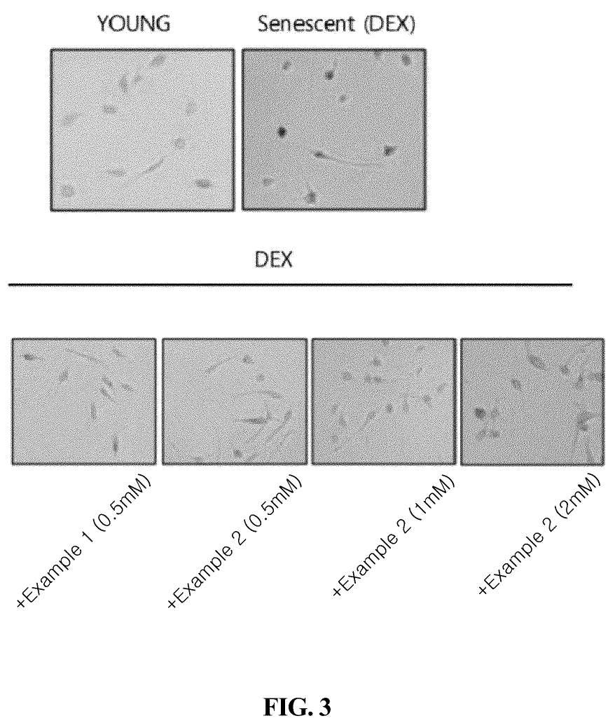

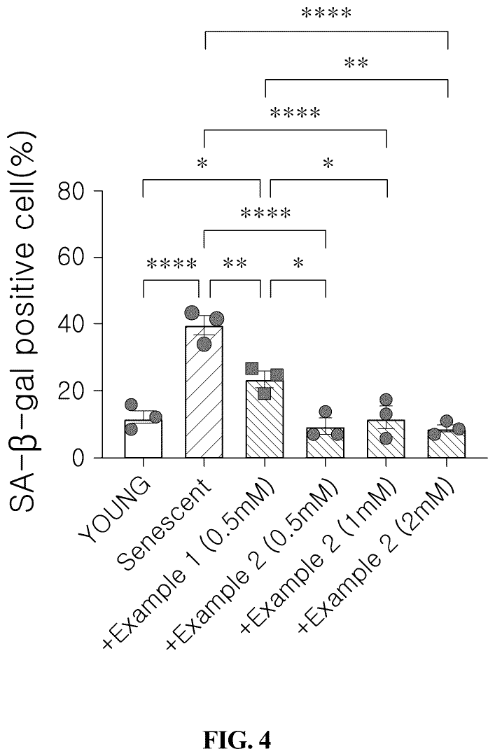

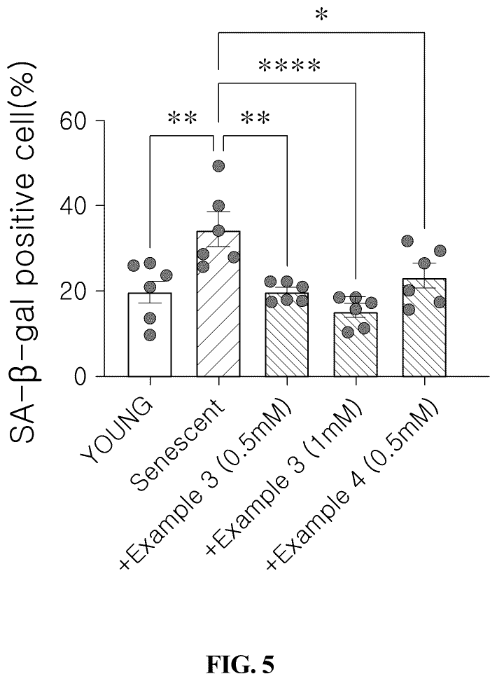

FIG. 3 shows images of control and experimental groups according to SA-β-gal staining for the compounds of the present invention, FIG. 4 shows the quantitative results according to SA-β-gal staining by concentration of the compounds of Example 1 and Example 2 of the present invention, and FIG. 5 shows the quantitative results according to SA-β-gal staining by concentration of the compounds of Example 3 and Example 4 of the present invention.

Using SA-β-gal staining, a senescence marker analysis method, to measure the proportion of senescent cells and evaluate the functional recovery ability of aged microglia by treatment with the compounds of the present invention, aged microglia (Senescent) induced by dexamethasone showed increased SA-β-gal, indicating cellular senescence. When the aged microglia were treated with compounds prepared in Examples 1, 2, 3, and 4 at various concentrations, all experimental groups showed decreasing SA-β-gal trends. Preferably, at the 0.5 mM concentration condition of Example 2 or the 1 mM concentration condition of Example 3, SA-β-gal reduction results were confirmed to levels equivalent to cells before senescence induction (YOUNG).

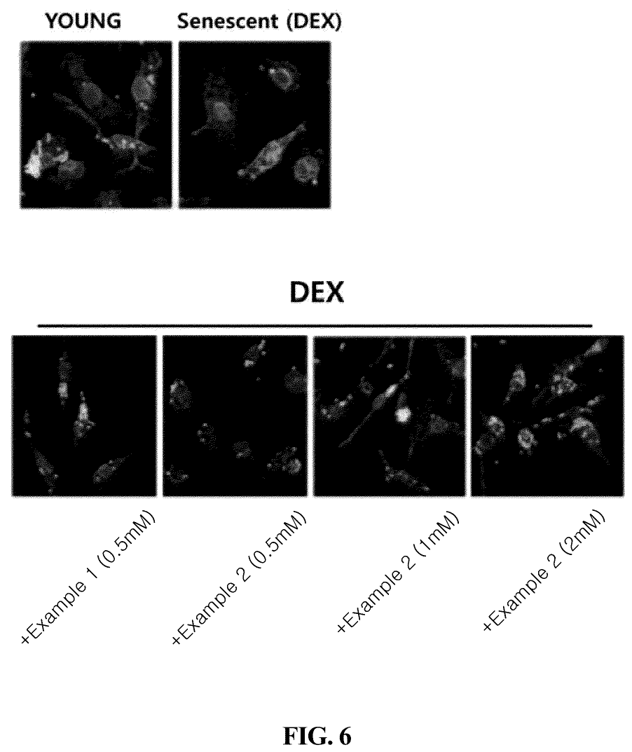

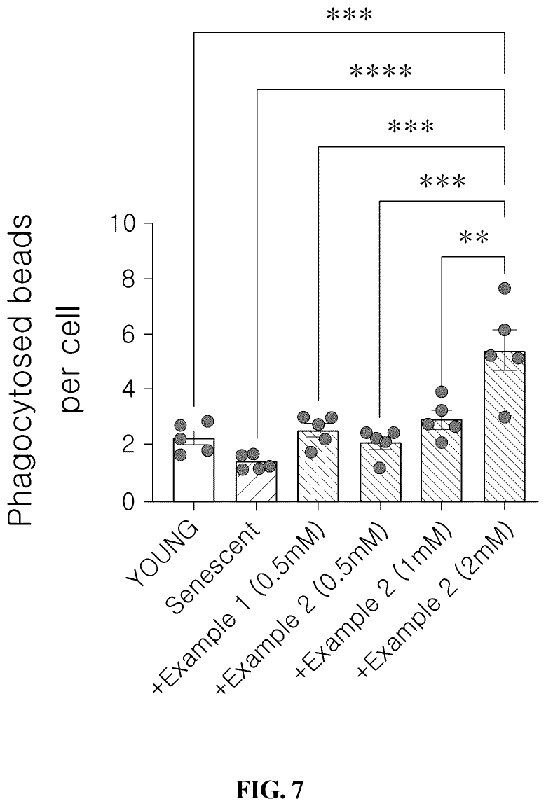

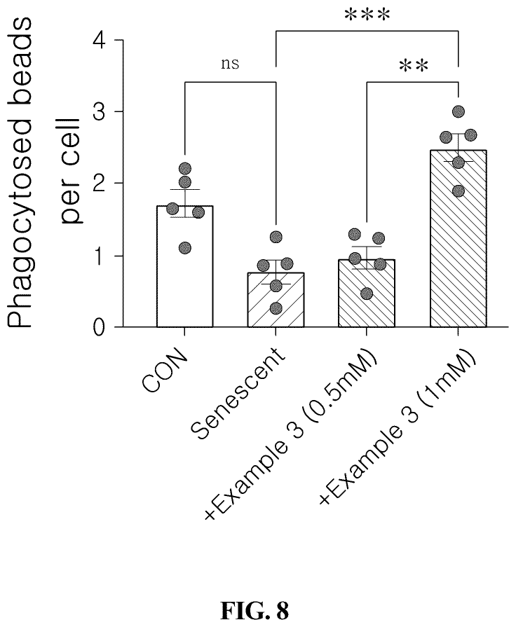

Additionally, primary cultured microglia were aged by treating with dexamethasone for 72 hours, then aged microglia were treated with the compounds of the present invention at various concentrations and phagocytosis activity recovery was evaluated. FIG. 6 shows images by experimental group for the brain immune cell phagocytic function recovery experiment for the compounds of the present invention, FIG. 7 shows the results on brain immune cell phagocytic function recovery by concentration of Examples 1 and 2 compounds of the present invention, and FIG. 8 shows the results of brain immune cell phagocytic function recovery by concentration of the compound of Example 3 of the present invention.

From these results, aged microglia (senescent) induced by dexamethasone showed decreased phagocytic function. In contrast, all experimental groups treated with compounds prepared in Examples 1 through 4 showed recovered phagocytic function. Particularly in the concentration range of 0.5 to 2 mM, concentration-dependent phagocytic function recovery or increase effects can be confirmed. More preferably, for the compound of Example 1, even at the low concentration condition of 0.5 mM, results confirmed that brain immune cell phagocytic function was recovered to levels above the control group (YOUNG).

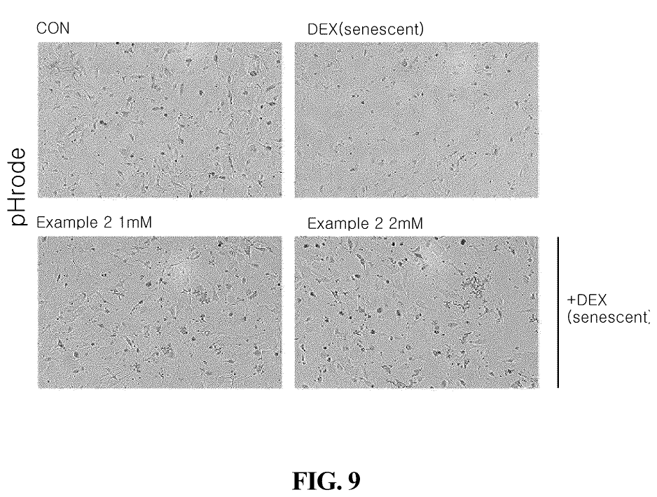

FIG. 9 is a live image of phagocytic function for zymosan by concentration of the compound of Example 2 of the present invention. Primary cultured microglia were aged by treating with dexamethasone for 72 hours, then the aged microglia were treated with the compound prepared in Example 2 at concentrations of 1 and 2 mM, and phagocytic function for zymosan was evaluated in real-time through live imaging equipment.

As a result, in the dexamethasone treatment group (DEX), fluorescent signals were clearly reduced, confirming decreased phagocytic function due to aging. When the aged microglia were treated with the compound prepared in Example 2 at various concentrations, red signals significantly increased compared to the dexamethasone treatment group (DEX), and particularly at 2 mM concentration, recovery to almost the same level as the control group (CON) was confirmed.

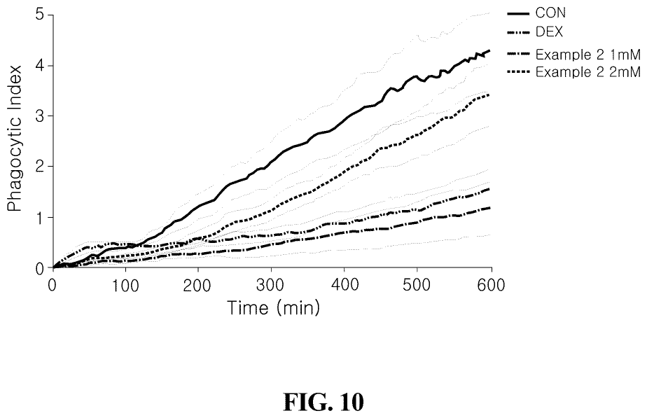

FIG. 10 shows the results of quantifying the phagocytic intensity over time for zymosan in FIG. 9. The control group (CON) rose to around 4.5-5 at approximately 600 minutes (10 hours), showing the highest phagocytic capacity, while the dexamethasone treatment group (DEX) generally showed low levels (˜2). When the compound prepared in Example 2 was treated at various concentrations, high concentration (2 mM) rose to around 4, showing intensity approaching CON, confirming improvement or recovery effects of functional decline in aged microglia.

Therefore, the compound of the present invention can lower cytotoxicity against aged microglia induced by dexamethasone, lower the expression of the senescence marker SA-β-gal (senescence associated-beta galactosidase), and at the same time, confirm the effect of recovering or increasing the phagocytic function of brain immune cells to the pre-aging state.

That is, from the results of recovery to the level of the pre-aging state, the present invention provides a composition for the rejuvenation of aged microglia containing the compound represented by Formula 1 or a pharmaceutically acceptable salt thereof.

Based on the above experimental results, the compound represented by Formula 1 or pharmaceutically acceptable salt thereof is contained at concentrations of 0.1 to less than 5 mM, preferably 0.5 to 2 mM. At this time, if it is less than 0.1 mM, the effect of the compound of the present invention on the functional recovery performance for aged microglia is low, and if it is 5 mM or more, there is a problem with cytotoxicity, which is not preferable.

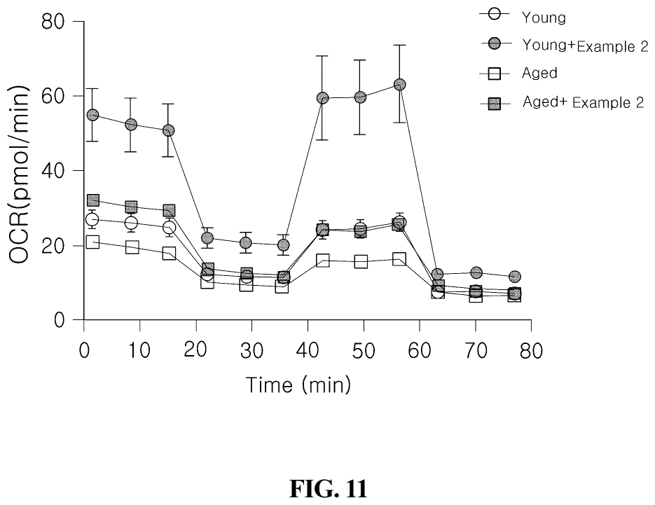

FIG. 11 shows oxygen consumption rate (OCR) results through Seahorse analysis by Example 2 compound of the present invention. For aged microglia (Aged, white square), mitochondrial function decreased, showing a result of reduced energy generation. On the other hand, in the aged mouse Example 2 compound treatment group (Aged+Example 2, gray square), OCR was recovered or higher to the level of young mouse microglia (Young, white circle). In particular, the group of young mouse treated with the compound of Example 2 (Young+Example 2, gray circle) exhibited a significantly increased OCR.

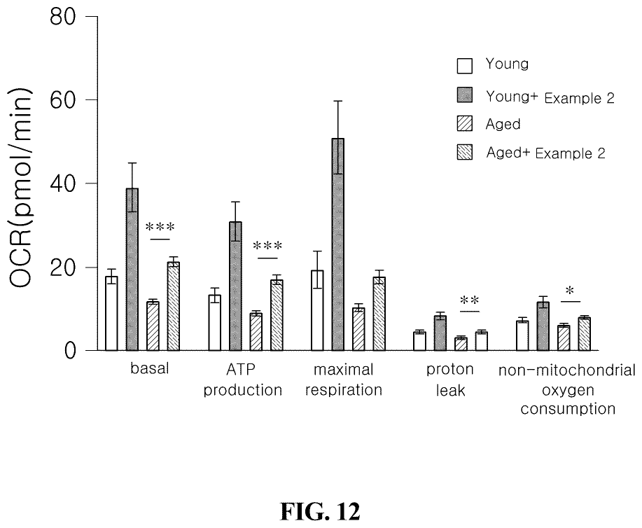

FIG. 12 shows the results on mitochondrial function recovery through the Seahorse analysis of FIG. 11. Specifically, through techniques evaluating mitochondrial metabolic function via basal respiration, ATP production, maximal respiration, proton leak, and non-mitochondrial oxygen consumption, aged microglia showed decreased mitochondrial function and reduced ATP production capacity, whereas the Example 2 compound treatment group (Aged+Example 2) showed results that restore mitochondrial function and regulate energy production and metabolism to normal levels.

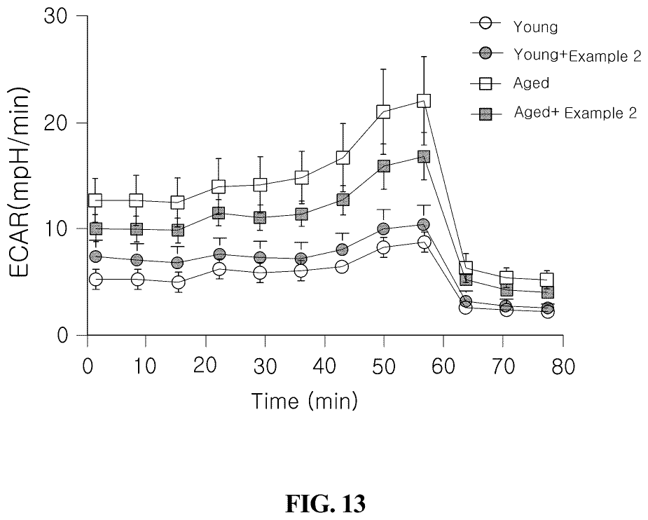

FIG. 13 shows the results of changes in cell metabolic function (ECAR) through cell metabolism analysis by the compound of Example 2 of the present invention. Young mouse microglia (Young, white circle) show low basic glycolysis activity and small metabolic changes. In contrast, aged microglia (Aged, white square) show much higher basic metabolic activity than young mouse microglia (Young, white circle) and large metabolic changes during aging. The aged microglia Example 2 compound treatment group (Aged+Example 2, gray square) shows that excessive metabolic responses of aged cells were partially suppressed by Example 2 compound treatment.

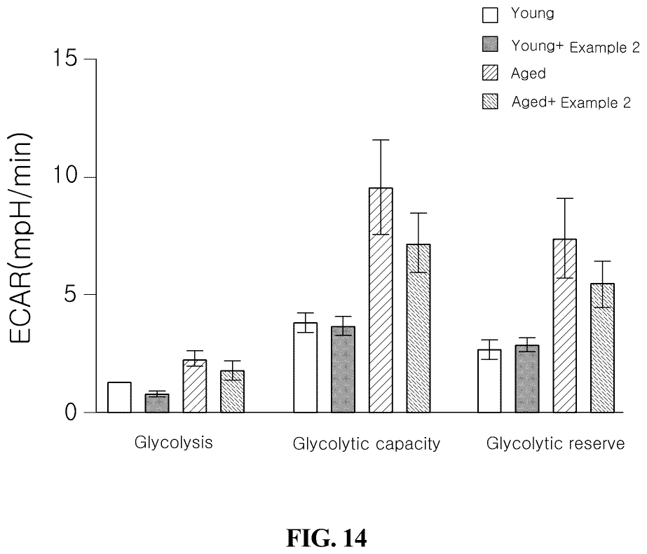

FIG. 14 shows quantitative results related to basic glycolysis from the analysis of FIG. 13. The aged microglia (Aged) group showed the highest results in glycolysis, glycolytic capacity, and glycolytic reserve, and decreased results by Example 2 compound treatment can be confirmed. From the above, it can be confirmed that the compound of the present invention can restore the mitochondrial function of microglia reduced due to aging and is effective in regulating cellular energy metabolism from the results of indicators related to basal glycolysis.

Therefore, the present invention provides a pharmaceutical composition preventing or treating brain diseases selected from the group consisting of neurodegenerative diseases, neuroinflammatory diseases, and neuropsychiatric disorders including depression, containing the compound represented by Formula 1 or pharmaceutically acceptable salt thereof.

The pharmaceutical composition for preventing or treating brain diseases of the present invention is effective for diseases having pathological characteristics of aged microglia. Specifically, the neurodegenerative diseases include Alzheimer's Disease, Parkinson's Disease, Huntington's Disease, ALS, and frontotemporal dementia. The neuroinflammatory diseases include Multiple Sclerosis, Stroke, and Encephalitis. Additionally, neuropsychiatric disorders include depression, anxiety disorders, obsessive-compulsive disorder, schizophrenia, or Autism Spectrum Disorder. More preferably, based on restoration of phagocytic function of aged microglia by the novel compounds of the present invention, it is useful as a pharmaceutical composition for preventing or treating any brain disease selected from the group consisting of Alzheimer's disease, Parkinson's disease, Huntington's disease, ALS, frontotemporal dementia, multiple sclerosis, and stroke.

The pharmaceutical composition containing the compound of Formula 1, stereoisomers, solvates, hydrates, or pharmaceutically acceptable salts thereof as an active ingredient can be formulated and used in the form of conventional pharmaceutical preparations. example, the pharmaceutical preparation may be prepared into various preparations for oral administration or parenteral administration, and the form of the preparation may be determined in various ways depending on the method of use, method of administration, purpose of administration, etc.

When manufactured as various formulations for oral or parenteral administration, formulation can be performed using one or more selected from the group consisting of diluents and excipients including fillers, bulking agents, binders, wetting agents, disintegrants, surfactants, etc., which are commonly used.

Solid formulations for oral administration may include tablets, pills, powders, granules, capsules, etc. Such solid formulations can be prepared by mixing the active ingredient with at least one or more excipients, for example, one or more selected from the group consisting of starch, calcium carbonate, sucrose, lactose, gelatin, etc. Additionally, lubricants such as magnesium stearate and talc can also be used besides simple excipients.

Liquid formulations for oral administration may include suspensions, internal solutions, emulsions, syrups, etc. When formulating as liquid formulations, water, which is a commonly used simple diluent, and/or liquid paraffin can be used, and optionally, one or more selected from the group consisting of various excipients such as wetting agents, sweeteners, fragrances, preservatives, etc., may be additionally included.

Parenteral administration can be performed by routes such as intravenous, intramuscular, subcutaneous, intraperitoneal, intranasal, transdermal administration. Formulations for parenteral administration include sterilized aqueous solutions, non-aqueous solutions, suspensions, emulsions, lyophilized formulations, suppositories, etc.

Non-aqueous solvents for preparing non-aqueous solutions or suspending agents for preparing suspensions include propylene glycol, polyethylene glycol, vegetable oils such as olive oil, injectable esters such as ethyl oleate, etc. As bases for suppositories, witepsol, macrogol, tween 61, cacao butter, laurin butter, glycerogelatin, etc. may be used.

Hereinafter, the present invention will be described in more detail through examples. These examples are for explaining the present invention in more detail, and the scope of the present invention is not limited to these examples.

### <Example 1> Synthesis of Compound of Formula 1-1

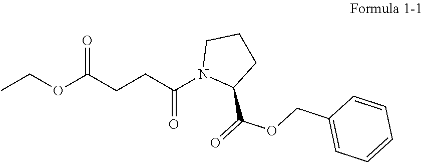 [SMILES: CCOC(=O)CCC(=O)N1CCC[C@H]1C(=O)OCc1ccccc1]

25 ml of dichloromethane was added to a 100 ml round bottom flask, a stirring bar was added, and 600 mg (2.48 mmol) of benzyl (2S)-pyrrolidine-2-carboxylate hydrochloride was dissolved therein. After slowly injecting 0.698 ml (4.96 mmol) of purified triethylamine, 0.489 ml (3.47 mmol) of ethyl 4-chloro-4-oxobutanoate was slowly added dropwise under a 0° C. water bath using ice water, followed by stirring at room temperature for 2 hours.

When the reaction was completed, the reaction was terminated with saturated sodium bicarbonate solution, extracted with 25 ml of dichloromethane and 25 ml of brine solution, and the dichloromethane layer was separated, dried over anhydrous magnesium sulfate, and then the solvent was removed using a vacuum evaporator.

The obtained compound was purified by silica gel column chromatography (n-hexane:ethyl acetate=1:1) and then evaporated under reduced pressure to obtain 774.12 mg (yield 90.0%) of the target compound ((S)-benzyl 1-(4-ethoxy-4-oxobutanoyl)pyrrolidine-2-carboxylate) represented by Formula 1-1. [Molecular Formula=C₁₈H₂₃NO₅, Molecular Weight=333.38, GC-MS: [C₄NH₈]⁺=70, [C₇H₇]⁺=91, [C₆H₉O₃]⁺=129, [C₁₀H₁₇NO₃]⁺=198, [C₁₆H₁₈NO₄]⁺=288, [M]⁺=333].

### <Example 2> Synthesis of Compound of Formula 1-2

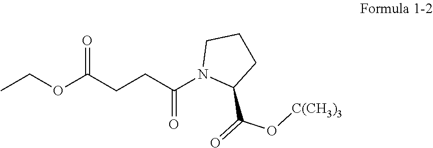 [SMILES: CCOC(=O)CCC(=O)N1CCC[C@H]1C(=O)OC(C)(C)C]

25 ml of dichloromethane was added to a 100 ml round bottom flask, a stirring bar was added, and 600 mg (2.89 mmol) of (S)-Pyrrolidine-2-carboxylic acid tert-butyl ester hydrochloride was dissolved therein.

After slowly injecting 0.812 ml (5.78 mmol) of purified triethylamine, 0.569 ml (4.05 mmol) of ethyl 4-chloro-4-oxobutanoate was slowly added dropwise under a 0° C. water bath using ice water, followed by stirring at room temperature for 2 hours.

When the reaction was completed, the reaction was terminated with saturated sodium bicarbonate solution, extracted with 25 ml of dichloromethane and 25 ml of brine solution, and the dichloromethane layer was separated, dried over anhydrous magnesium sulfate, and then the solvent was removed using a vacuum evaporator.

The obtained compound was purified by silica gel column chromatography (n-hexane:ethyl acetate=1:1) and then evaporated under reduced pressure to obtain 734.50 mg (yield 84.9%) of the target compound ((S)-tert-butyl 1-(4-ethoxy-4-oxobutanoyl) pyrrolidine-2-carboxylate) represented by Formula 1-2. [MF=C₁₅H₂₅NO₅, MW=299.36, GC-MS=[C₄NH₈]⁺=70, [C₅H₇NO]⁺=97, [C₆H₉O₃]⁺=129, [C₁₀H₁₇NO₃]⁺=198, [C₁₃H₂₀NO₄]⁺=254, [M]⁺=299].

### <Example 3> Synthesis of Compound of Formula 1-3

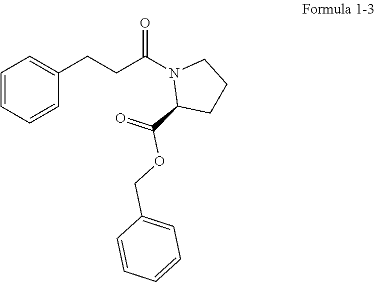 [SMILES: O=C(OCc1ccccc1)[C@@H]1CCCN1C(=O)CCc1ccccc1]

25 ml of dichloromethane was added to a 100 ml round bottom flask, a stirring bar was added, and 600 mg (2.48 mmol) of benzyl (2S)-pyrrolidine-2-carboxylate hydrochloride was dissolved therein. After slowly injecting 0.812 ml (5.78 mmol) of purified triethylamine, 0.440 ml (2.98 mmol) of 3-phenylpropionyl chloride was slowly added dropwise under a 0° C. water bath using ice water, followed by stirring at room temperature for 2 hours.

When the reaction was completed, the reaction was terminated with saturated sodium bicarbonate solution, extracted with 25 ml of dichloromethane and 25 ml of brine solution, and the dichloromethane layer was separated, dried over anhydrous magnesium sulfate, and then the solvent was removed using a vacuum evaporator.

The obtained compound was purified by silica gel column chromatography (ethyl acetate:n-hexane=1:1) and then evaporated under reduced pressure to obtain 826.3 mg (yield 95.5%) of the target compound ((S)-Benzyl 1-(3-phenylpropanoyl) pyrrolidine-2-carboxylate) represented by Formula 1-3. [MW=337.41, GC-MS=[C₄NH₈]⁺=70, [C₇H₇]⁺=91, [C₅H₈NO₂]⁺=114, [C₁₂H₁₄NO₂]⁺=202, [C₁₃H₁₆NO₃]⁺=246, [M]⁺=337].

### <Example 4> Synthesis of Compound of Formula 1-4

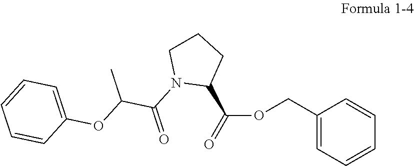

25 ml of dichloromethane was added to a 100 ml round bottom flask, a stirring bar was added, and 600 mg (2.48 mmol) of benzyl (2S)-pyrrolidine-2-carboxylate hydrochloride was dissolved therein. After slowly injecting 0.698 ml (4.96 mmol) of purified triethylamine, 0.466 ml (2.98 mmol) of ethyl 2-phenoxypropionyl chloride was slowly added dropwise under a 0° C. water bath using ice water, followed by stirring at room temperature for 2 hours.

When the reaction was completed, the reaction was terminated with saturated sodium bicarbonate solution, extracted with 25 ml of dichloromethane and 25 ml of brine solution, and the dichloromethane layer was separated, dried over anhydrous magnesium sulfate, and then the solvent was removed using a vacuum evaporator.

The obtained compound was purified by silica gel column chromatography (ethyl acetate:n-hexane=1:1) and then evaporated under reduced pressure to obtain 807.1 mg (yield 92.2%) of the target compound ((2S)-benzyl 1-(2-phenoxypropanoyl) pyrrolidine-2-carboxylate) represented by Formula 1-4. [MF=C₂₁H₂₃NO₄, MW=353.15, GC-MS=[C₄NH₈]⁺=70, [C₇H₇]⁺=91, [CH₉O]⁺=121, [C₁₃H₁₆NO₂]⁺=218, [C₁₃H₁₆NO₃]⁺=260, [M]⁺=353].

### <Example 5> Synthesis of Compound of Formula 1-5

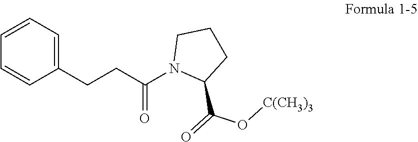 [SMILES: CC(C)(C)OC(=O)[C@@H]1CCCN1C(=O)CCc1ccccc1]

25 ml of dichloromethane was added to a 100 ml round bottom flask, a stirring bar was added, and 500 mg (2.40 mmol) of t-butyl (2S)-pyrrolidine-2-carboxylate hydrochloride was dissolved therein. After slowly injecting 0.698 ml (4.96 mmol) of purified triethylamine, 0.426 ml (2.88 mmol) of 3-phenylpropionyl chloride was slowly added dropwise under a 0° C. water bath using ice water, followed by stirring at room temperature for 2 hours.

When the reaction was completed, the reaction was terminated with saturated sodium bicarbonate solution, extracted with 25 ml of dichloromethane and 25 ml of brine solution, and the dichloromethane layer was separated, dried over anhydrous magnesium sulfate, and then the solvent was removed using a vacuum evaporator.

The obtained compound was purified by silica gel column chromatography (ethyl acetate:n-hexane=1:1) and then evaporated under reduced pressure to obtain 698.3 mg (yield 95.8%) of the target compound (S)-tert-butyl 1-(3-phenylpropanoyl) pyrrolidine-2-carboxylate) represented by Formula 1-5. [MF=C₁₈H₂₅NO₃, MW=303.40, [C₄H₉]⁺=70, [C₇H₇]⁺=91, [C₈H₉]⁺=105, [C₁₃H₁₇NO]⁺=202, [M]⁺=303].

### <Example 6> Synthesis of Compound of Formula 1-6

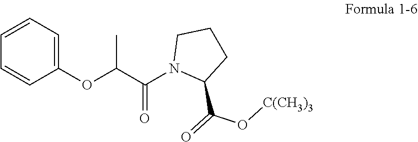 [SMILES: CC(Oc1ccccc1)C(=O)N1CCC[C@H]1C(=O)OC(C)(C)C]

25 ml of dichloromethane was added to a 100 ml round bottom flask, a stirring bar was added, and 500 mg (2.40 mmol) of t-butyl (2S)-pyrrolidine-2-carboxylate hydrochloride was dissolved therein. After slowly injecting 0.698 ml (4.96 mmol) of purified triethylamine, 0.452 ml (2.89 mmol) of 2-phenoxypropionyl chloride was slowly added dropwise under a 0° C. water bath using ice water, followed by stirring at room temperature for 2 hours.

When the reaction was completed, the reaction was terminated with saturated sodium bicarbonate solution, extracted with 25 ml of dichloromethane and 25 ml of brine solution, and the dichloromethane layer was separated, dried over anhydrous magnesium sulfate, and then the solvent was removed using a vacuum evaporator.

The obtained compound was purified by silica gel column chromatography (ethyl acetate:n-hexane=1:1) and then evaporated under reduced pressure to obtain 642.7 mg (yield 83.8%) of the target compound ((2S)-tert-butyl 1-(2-phenoxypropanoyl) pyrrolidine-2-carboxylate) represented by Formula 1-6. [Molecular formula=C₁₈H₂₅NO₄, molecular weight=319.40, [C₄H₈N]⁺=70, [CH₉O]⁺=121, [C₁₃H₁₆NO₂]⁺=218, [M]⁺=319].

### <Example 7> Synthesis of Compound of Formula 1-7

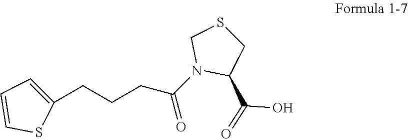 [SMILES: O=C(O)[C@@H]1CSCN1C(=O)CCCc1cccs1]

30 ml of THF solvent was added to a 100 ml round bottom flask, a stirring bar was added, and 500 mg (3.75 mmol) of 4R-1,3-thiazolidine-4-carboxylic acid was dissolved therein.

After slowly injecting 1.056 ml (7.50 mmol) of purified triethylamine, 0.452 ml (4.50 mmol) of 4-(thiophene-2-yl) butanoyl chloride was slowly added dropwise under a 0° C. water bath using ice water, followed by stirring at room temperature for 2 hours.

When the reaction was completed, 10 ml of water was added to terminate the reaction, extracted with 50 ml of diethyl ether and 50 ml of brine solution, and the diethyl ether layer was separated, dried over anhydrous magnesium sulfate, and then the solvent was removed using a vacuum evaporator.

The obtained compound was purified by silica gel column chromatography (ethyl acetate:n-hexane=1:1) and then evaporated under reduced pressure to obtain 891.0 mg (yield 83.3%, MW=285.05) of the target compound ((R)-3-(4-thiophene-2-yl) butanoyl)thiazolidine-4-carboxylic acid) represented by Formula 1-7.

TABLE 1

| | Formula 1 | | | |
|---|---|---|---|---|
| | R′ | R″ | X | MW |
| Example 1 | benzyl | —CH₂CH₂COOCH₂CH₃ | —CH₂— | 333 |
| Example 2 | t-butyl | —CH₂CH₂COOCH₂CH₃ | —CH₂— | 299 |
| Example 3 | benzyl | —CH₂CH₂C₆CH₅ | —CH₂— | 337 |
| Example 4 | benzyl | —CH₂(CH₃)OC₆CH₅ | —CH₂— | 353 |
| Example 5 | t-butyl | —CH₂(CH₃)OC₆CH₅ | —CH₂— | 303 |
| Example 6 | t-butyl | —CH₂(CH₃)OC₆CH₅ | —CH₂— | 319 |
| Example 7 | —H | —(CH₂)₃-(2{-thienyl) | S | 285 |
| Example 8 | —H | —CH₂CH₂OCH₃ | S | 219 |
| Example 9 | benzyl | —CH₂CH₂OCH₃ | —CH₂— | 291 |
| Example 10 | —H | —CH₂CH₂OCH₃ | —CH₂— | 201 |
| Example 11 | t-butyl | —CH₂CH₂OCH₃ | —CH₂— | 257 |
| Example 12 | —H | —CH₂CH₂COOCH₂CH₃ | S | 261 |
| Example 13 | —H | —CH₂CH₂COOCH₂CH₃ | —CH₂— | 243 |
| Example 14 | —H | —C(OH)(CH₃)(CF₃) | S | 289 |
| Example 15 | benzyl | —C(OH)(CH₃)(CF₃) | —CH₂— | 361 |
| Example 16 | —H | —C(OH)(CH₃)(CF₃) | —CH₂— | 271 |
| Example 17 | t-butyl | —C(OH)(CH₃)(CF₃) | —CH₂— | 327 |
| Example 18 | benzyl | —(CH₂)₃-(2{-thienyl) | —CH₂— | 357 |
| Example 19 | —H | —(CH₂)₃-(2{-thienyl) | —CH₂— | 367 |
| Example 20 | t-butyl | —(CH₂)₃-(2{-thieny1) | —CH₂— | 323 |
| Example 21 | —H | —(CH₂)₃N(CH₃)₂ | S | 246 |
| Example 22 | benzyl | —(CH₂)₃N(CH₃)₂ | —CH₂— | 318 |
| Example 23 | —H | —(CH₂)₃N(CH₃)₂ | —CH₂— | 228 |
| Example 24 | t-butyl | —(CH₂)₃N(CH₃)₂ | —CH₂— | 284 |
| Example 25 | —H | —CH₂CH₂C₆CH₅ | S | 265 |
| Example 26 | —H | —CH₂CH₂C₆CH₅ | —CH₂— | 247.3 |
| Example 27 | —H | —CH₂(CH₃)OC₆CH₅ | S | 281.3 |
| Example 28 | —H | —CH₂(CH₃)OC₆CH₅ | —CH₂— | 263.3 |

### <Experimental Example 1> Microglia Culture and Isolation

To obtain microglia as experimental materials, 13.5-day mouse embryos were isolated from the uterus, the neuroepithelial layer of the head was dissected to prepare single-cell suspensions in HBSS (Gibco, 14170-112). Cells in HBSS were centrifuged at 1200 rpm for 3 minutes, then HBSS was removed. 1× Trypsin-EDTA (Gibco, 15400-054) was added and incubated in a water bath for 3 minutes, then centrifuged again.

The precipitated cells were resuspended in DMEM medium containing 1000 FBS (Gibco, #16000-044), 0.1×GlutaMAX (Gibco, #11995-065), 1% penicillin/streptomycin (Gibco, #15140122). Cultured in 25T flasks coated with Poly-D-lysine (Sigma, #P7280) for 2 weeks. Subsequently, subculture was performed and cultured for an additional week. Finally, microglia were isolated through MACS separation method using CD11b (Miltenyi, #130-093-636) beads and MS columns (Milteyni, #130-042-201).

### <Experimental Example 2> Evaluation of Cytotoxicity of Drugs

To confirm cytotoxicity when applying the compounds of Examples 1 through 4 as drugs, experiments were conducted using the MTT (Dogen #EZ-1000) method. The compounds to be treated on cells were prepared at concentrations of 0.5, 1, 2, and 5 mM for each compound prepared in Examples 1 through 4. Cytotoxicity experiments confirmed the cytotoxicity of the compounds applied to microglia and the cytotoxicity of the compounds on aged microglia induced by 5 nM dexamethasone, respectively.

The specific experimental method was as follows: Isolated microglia were cultured with culture medium (DMEM+FBS+Penicillin/streptomycin+glutamax) in 96-well plates at 37° C. for one day. The cultured microglia were treated with the compounds prepared in Examples 1 through 4 diluted in culture medium at concentrations of 0.5, 1, 2, and 5 mM in equal volumes and cultured for 24 hours. 10 μl of EZ-CYTOX reagent was treated to each well of the microglia treated with the compound and reacted for 2 hours and 30 minutes. The treated plate was gently mixed for about 1 minute before measurement, and absorbance was measured at 450 nm.

Additionally, the cytotoxicity experiment on aged microglia was performed by culturing microglia in the same culture medium at 37° C. for one day, then aging them by treating with 5 nM dexamethasone for 24 hours at 37° C. The culture medium was removed, and each compound from Examples 1 through 4 was diluted in culture medium at various concentrations, treated in equal volumes, and cultured for 24 hours. 10 μl of EZ-CYTOX reagent was treated to each well of the microglia treated with the compound and reacted at 37° C. for 2 hours and 30 minutes. The treated plate was gently mixed for about 1 minute before measurement, and absorbance was measured at 450 nm.

FIG. 1 shows the cytotoxicity experimental results of primary cultured microglia using the compounds of Examples 1 and 2 of the present invention. When the compounds of Example 1 and Example 2 were applied as drugs to aged microglia induced by 5 nM dexamethasone treatment, the decrease in cell survival rate was partially recovered. In particular, a tendency to maintain or increase cell viability was confirmed with increasing concentration in the drug concentration range of 0.5 to 2 mM.

FIG. 2 shows the cytotoxicity experimental results of primary cultured microglia using the compounds of Examples 3 and 4 of the present invention. It can be confirmed that the compounds of Example 3 and Example 4 have an effect of inhibiting the decrease in cell viability, with viability increasing effects in the concentration range of 0.5 to 2 mM, particularly remarkable at 1 to 2 mM.

### <Experimental Example 3> Evaluation Through Cell Senescence Marker Staining Method (SA-β-Gal Staining)

To confirm the functional recovery ability of the compounds of Examples 1 through 4 on aged microglia, SA-β-gal staining was performed. Primary cultured microglia were cultured at 1×10⁵ per well in 24-well plates. Wells excluding the control group were treated with 5 nM dexamethasone for 3 days, and the culture medium was removed. The microglia with removed medium were treated with the compounds of Examples 1 through 4 diluted in medium according to concentrations of 0.5, 1, 2, and 5 mM in equal volumes and cultured for 24 hours.

The microglia cultured for 24 hours had their medium removed and were washed 3 times for 1 minute each using PBS. The washed microglia were treated with staining solution prepared using a beta-galactosidase activation kit (Cell biolabs, #CBA-230) added to the cells, then incubated at 37° C. for 4 hours. After washing the cells with PBS, cells-stained blue were observed using a microscope.

FIG. 3 shows images of control and experimental groups according to SA-β-gal staining for the compounds of the present invention.

FIG. 4 shows the quantitative results according to SA-β-gal staining by concentration of Examples 1 and 2 compounds of the present invention. The dexamethasone treatment group (Senescent) showed increased SA-β-gal, indicating cellular senescence. On the other hand, when the compounds of Examples 1 and 2 were applied to the aged microglia, a decreasing tendency of SA-β-gal was observed. In particular, the 0.5 mM concentration condition of the compound of Example 2 confirmed an SA-β-gal result lower than that of the cells before senescence induction (YOUNG). FIG. 5 shows the quantitative results according to SA-β-gal staining by concentration of Examples 3 and 4 compounds of the present invention. When the compounds of Example 3 and Example 4 were applied compared to the dexamethasone treated group (Senescent), SA-β-gal reduction results were shown, and it was confirmed to be at the level of the control group (CON) before senescence induction. In particular, the 1 mM concentration condition of the Example 3 compound showed SA-β-gal results at levels lower than CON.

### <Experimental Example 4> Evaluation of Brain Immune Cell Phagocytosis Activity

To confirm the functional recovery ability of the compounds prepared in Examples 1 to 4 on aged microglia, phagocytic function was evaluated. Primary cultured microglia were cultured with 12 mm coverslips placed in 24-well plates at 1×10⁵ cells per well. The cultured cells were treated with 5 nM dexamethasone for 3 days, and the culture medium was removed. The microglia with removed medium were treated with the compounds of Examples 1 to 4 diluted in medium according to concentrations of 0.5, 1, 2, and 5 mM in equal volumes for 24 hours.

Latex microbeads were added to each well of the microglia cultured for 24 hours, gently mixed, wrapped in foil, and reacted at 37° C. for 2 hours. Cells were cultured for 2 hours at 37° C. with 2 μl of fluoresbrite microspheres (Sigma, #L3030) containing red fluorescent latex microbeads. To stop phagocytic function, 2 ml of ice-cold PBS was added. Cells were washed twice and fixed with 4% paraformaldehyde solution. Cells were washed twice with PBS, and then stained for IBA1, a microglia marker, overnight. After staining with secondary antibody, cells were washed twice with PBS, fixed using mounting solution containing DAPI, and when fixation was completed, analyzed using confocal microscopy. The level of phagocytic function activity was confirmed through the number of phagocytosed beads per cell.

FIG. 6 shows images by experimental group for brain immune cell phagocytic function recovery experiments with the compounds of the present invention, and FIG. 7 shows the results on brain immune cell phagocytic function recovery by concentration of Examples 1 and 2 compounds of the present invention. Aged microglia (senescent) by dexamethasone treatment showed decreased phagocytosis (phagocytosed beads per cell). On the other hand, as a result of culturing the aged microglia with the compounds prepared in Examples 1 and 2 at various concentrations, they recovered to levels above the control group (YOUNG), and particularly the 2 mM concentration of Example 2 was confirmed at 2.5 times higher levels.

FIG. 8 shows the results on brain immune cell phagocytic function recovery by concentration of the Example 3 compound of the present invention. Even at the 0.5 mM concentration condition of Example 3, phagocytic function was recovered compared to aged microglia (senescent), and particularly at the 1 mM concentration condition, it was higher than aged microglia (senescent) and further showed results at twice the level of the control group (YOUNG).

From the above, it was confirmed that the compound of the present invention has recovery performance for brain immune cell phagocytosis activity against aged microglia.

Additionally, for live imaging evaluation of phagocytosis, primary cultured microglia were aged by treating with dexamethasone for 72 hours, then after treating with the compound prepared in Example 2 at concentrations of 1 and 2 mM, phagocytic function for zymosan was evaluated through live cell imaging equipment. Specifically, primary microglia were cultured at a density of 6×10⁴ cells/ml in 48-well plates. 4 [symbol] of pHrodo-conjugated zymosan was treated to cells, and real-time imaging was performed for a total of 10 hours at 5-minute intervals using Juli Stage equipment. The phagocytic index represents the ratio of cells showing red fluorescence among total cells by detecting that pHrodo-zymosan is contained in autophagic lysosomes within cells and exhibits red fluorescence in low pH environments.

From the live image of FIG. 9, internalization of red fluorescent particles (pHrodo-labeled zymosan) appeared clearly in the control group (CON), confirming that phagocytic ability was generally active. In the dexamethasone treated group (DEX), the fluorescent signal was significantly reduced, confirming that phagocytic function was significantly degraded due to aging. In the concentration-dependent treatment groups of the compound prepared in Example 2, red signals significantly increased compared to the dexamethasone treatment group (DEX), and particularly at 2 mM concentration, recovery to almost the same level as the control group (CON) was observed.

FIG. 10 shows the results of quantifying phagocytic intensity over time for zymosan. The control group (CON) showed the highest phagocytic ability by rising to near 4.5-5 at about 600 minutes (10 hours), while the dexamethasone treatment group (DEX) remained at generally low levels (˜2), confirming that phagocytic activity was suppressed by senescence induction. On the other hand, in the concentration-dependent treatment groups of the compound prepared in Example 2, the index steadily increased over time, and particularly high concentration (2 mM) rose to around 4, showing intensity approaching CON, confirming improvement or recovery effects of functional decline in aged microglia.

### <Experimental Example 5> Cell Metabolism Analysis

Step 1: Direct Isolation Method of Microglia from Mice

Aged microglia were isolated from 85-90-week-old male C57BL/6J mice. Direct isolation of microglia was performed by enzymatic cell dissociation method using the Adult Brain Dissociation Kit (130-107-677, Miltenyi Biotec) according to the manufacturer's instructions. Brain tissue pieces (maximum 500 mg) were transferred to GentleMACS C tubes (130-096-334, Miltenyi Biotec) containing 1950 [symbol] of enzyme mix 1 (enzyme P and buffer Z). Subsequently, 30 μl of enzyme mix 2 (enzyme A and buffer Y) was added.

The GentleMACS C tube was tightly closed and placed in an inverted position in the sleeve of a dissociator with heater (GentleMACS Octo Dissociator, 130-096-427, Miltenyi Biotec), then the GentleMACS program (37C_ABDK_01) was run. After dissociation, samples were briefly centrifuged to collect samples at the bottom of the tube, then filtered through a 70 m strainer (130-098462, Miltenyi Biotec), washed with DPBS, and centrifuged again. The resulting pellet was resuspended in cold DPBS, myelin and cell debris were removed with debris removal solution, and red blood cells were removed through red blood cell removal solution. Finally, microglia were magnetically isolated using microbeads conjugated to anti-CD11b mAb as previously described.

### Step 2: Cell Metabolism Analysis (Seahorse Analysis)

Brains of 11-week-old mice (YOUNG) and 85-90 week-old mice (AGED) were isolated and homogenized using the Adult Brain Dissociation Kit (Miltenyi, #130-107-677) according to manufacturer's instructions. Microglia were isolated through MACS separation method using CD11b (Miltenyi, #130-093-636) beads and MS columns (Milteyni, #130-042-201). Cells were dispensed into Seahorse culture plates coated with Poly-D-lysine (Sigma, #P7280) at 2×10⁵ cells in 80 μl of medium per well and cultured. Microglia were cultured for 5 days, and drugs were treated on the 5th day.

On the 6th day, the metabolic profile of cells was analyzed using a Seahorse Extracellular Flux (XF96) Analyzer. Sensor cartridges used for real-time metabolic analysis of cells were hydrated overnight at 37° C. in a CO₂-free incubator by adding Seahorse XF Calibrant solution to each well of the utility plate. Before experiments, cells were washed twice with experimental medium suitable for Seahorse analysis, then medium was added to 180 μl per well and cultured in a CO₂-free incubator.

For the mitochondrial stress test, oligomycin 1.5 μM, carbonyl cyanide-4-(trifluoromethoxy)phenylhydrazone 2.0 μM, and antimycin 0.5 μM were loaded in ports to be sequentially injected. For the glycolytic stress test, glucose 10 mM, oligomycin 20 PM, and 2-deoxy-D-glucose 50 mM were loaded in ports. After calibration, oxygen consumption rate and extracellular acidification rate were measured every 8 minutes for 96 minutes, and reagents were sequentially injected at 24-minute intervals. Oxygen consumption rate and extracellular acidification rate were automatically calculated using Seahorse XF96 software, and 3 to 6 repeated experiments were performed for each sample.

To evaluate the effects of the compound of Example 2 on mitochondrial function and metabolism of aged microglia and confirm metabolic function recovery ability, Seahorse analysis was performed.

FIG. 11 shows oxygen consumption rate (OCR) results through Seahorse analysis by Example 2 compound of the present invention. Compared to young mouse microglia (Young, white circle), aged microglia (Aged, white square) showed overall decreased OCR, that is, confirmed mitochondrial function decline. On the other hand, even in the aged microglia Example 2 compound treatment group (Aged+Example 2, gray square), OCR was recovered or higher to the level of young mouse microglia (YOUNG). In particular the group treated with the compound of Example 2 in the young mice (Young+Example 2, gray circle) confirmed remarkably increased OCR increase effects compared to young mouse microglia (YOUNG) baseline.

FIG. 12 shows the results on mitochondrial function recovery through the Seahorse analysis of FIG. 11. Specifically, as a technique for evaluating mitochondrial metabolic function, it was confirmed how energy production and metabolism of microglia change due to aging through basal respiration, ATP production, maximal respiration, proton leak, and non-mitochondrial oxygen consumption.

Specifically, basal respiration decreased in aged mice (Aged), but basal respiration was recovered to young mouse levels in the Example 2 compound treatment group (Aged+Example 2). Additionally, aged microglia showed decreased ATP production capacity, but the Example 2 compound treatment group (Aged+Example 2) showed increased ATP production results, suggesting the possibility of cellular energy production recovery. Also, maximal respiration was greatly decreased in aged microglia, and the group treated with the compound of Example 2 (Aged+Example 2) confirmed a tendency to recover to the level of young mice.

As an indicator of mitochondrial dysfunction, energy inefficiency increased in aged microglia in the results of proton leak, whereas the possibility of improving mitochondrial function was confirmed in the group treated with the compound of Example 2 (Aged+Example 2). Additionally, non-mitochondrial oxygen consumption results showed an increase in aged cells, meaning inefficient energy use, whereas it significantly decreased in the group treated with the compound of Example 2 (Aged+Example 2), confirming that mitochondrial metabolism is regulated more efficiently.

FIG. 13 shows the result of changes in cellular metabolic function (Extracellular acidification rate: ECAR) through Seahorse analysis by the Example 2 compound of the present invention. Microglia of young mice (Young, white circle) maintained a low ECAR of 6 mpH/min during Basal (0-40 min) and then had no rapid change, confirming that young microglia have low basal glycolysis activity and metabolic changes are not large.

In the microglia of the young mice, the group treated with the compound of Example 2 (Young+Example 2, gray circle) was slightly increased glycolysis due to Example 2 compound treatment but did not show strong reactions as in aged cells. On the other hand, aged microglia (Aged, white square) showed high ECAR (9 mpH/min) from Basal (0-40 minutes), rapidly increased to 25 mpH/min at Peak value (around 50 minutes) after stimulation, then showed rapid decrease results, confirming that basic metabolic activity is much higher than young mouse microglia (YOUNG) and metabolic changes are large during aging.

The aged microglia Example 2 compound treatment group (Aged+Example 2, gray square) showed similar Basal levels to AGED, but Peak value after stimulation was slightly lower (˜20 mpH/min), supporting that excessive metabolic responses of aged cells were partially suppressed due to Example 2 compound treatment.

FIG. 14 shows quantitative results related to basic glycolysis from the analysis of FIG. 13. Glycolysis was highest in the AGED group, and the groups treated with the compound of Example 2 (Young+Example 2, Aged+Example 2) did not significantly affect basic glycolysis itself. For glycolytic capacity and glycolytic reserve, Young and Young+Example 2 showed relatively low levels, whereas the Aged group had the highest value, and a result of slight decrease due to the treatment with the compound of Example 2 was confirmed.

From the above, although the present invention has been described in detail only with respect to the described embodiments, it is obvious to those skilled in the art that various modifications and variations are possible within the technical scope of the present invention, and such modifications and variations naturally belong to the appended claims.

What is claimed is:

1. A compound represented by the following Formula 1 or a pharmaceutically acceptable salt thereof for use in a composition for rejuvenating aged microglia: 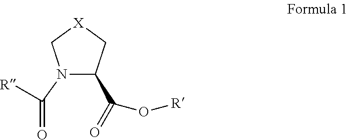
wherein in Formula 1,
R′ is hydrogen, phenyl, benzyl, or C₁-C₄ linear or branched alkyl;
R″ is a C₁-C₆ alkyl group unsubstituted or substituted with hydroxy (—OH), halogen, C₁-C₄ alkoxy, C₁-C₄ alkylamino or di(C₁-C₄) alkylamino, alkoxycarbonyl, phenyl, phenoxy, or a 5- to 6-membered heteroaryl containing N, O, or S; and
X is any one selected from the group consisting of CH₂—, —S—, —O—, —NH—, or —N(C₁-C₄ alkyl) and —C═O.

2. The compound or a pharmaceutically acceptable salt thereof according to claim 1, wherein the compound has a low molecular weight capable of penetrating the Blood-Brain Barrier (BBB).

3. The compound or a pharmaceutically acceptable salt thereof according to claim 2, wherein the compound has a weight average molecular weight (MW) of 800 g/mol or less.

4. A pharmaceutical composition for preventing or treating brain diseases containing the compound or a pharmaceutically acceptable salt thereof according to claim 1.

5. The pharmaceutical composition for preventing or treating brain diseases according to claim 4, wherein the compound or pharmaceutically acceptable salt thereof is contained at a concentration of 0.1 to less than 5 mM.

6. The pharmaceutical composition for preventing or treating brain diseases according to claim 4, wherein the brain disease is any one selected from the group consisting of Alzheimer's Disease, Parkinson's Disease, Huntington's Disease, Lou Gehrig's disease (ALS), Frontotemporal Dementia, Multiple Sclerosis, and Stroke.
